(19) United States
(12) Patent Application Publication
(10) Pub. No.: US 2019/0175065 A1
(43) Pub. Date: Jun. 13, 2019

(54) SPIROERGOMETRY APPARATUS

(71) Applicant: Markus KNESTEL, Hopferbach (DE)

(72) Inventor: Markus KNESTEL, Hopferbach (DE)

(21) Appl. No.: 16/324,402

(22) PCT Filed: Aug. 8, 2017
(86) PCT No.: PCT/EP2017/070097

§ 371 Date: Feb. 8, 2019

(30) Foreign Application Priority Data
Aug. 8, 2016 (DE) 10 2016 214 702.1

(51) Int. Cl. A61B 5/083; A61B 5/00; A61B 5/087; A61B 5/091; A61B 5/097

(52) U.S. Cl. CPC A61B 5/0836; A61B 5/0075; A61B 5/0878; A61B 2560/0214; A61B 5/0833; A61B 5/097; A61B 5/0873; A61B 5/091

(57) ABSTRACT

A spiroergometry apparatus for detecting parameters of a respiratory gas. The spiroergometry apparatus has a main body, a measuring device, a computing unit and an energy store. The measuring device includes a sensor, which is provided on the main body, whereby it becomes possible to detect parameters directly in the respiratory gas flow, so that an in-situ detection of the parameters of the respiratory gas is made possible.

The present invention relates to a spiroergometry apparatus for detecting parameters of a respiratory gas, such as the O₂ concentration or the CO₂ concentration.

Mobile ergospirometry devices, e.g. for stress examinations in humans, have been known for several years. With the help of such systems, analyses can be carried out directly at the sports area or workplace under natural conditions and in stress situations. Telemetry units are used to transmit measurement data in real time to a personal computer or notebook, the training or exercise process being controllable accordingly after evaluating the data on the personal computer. Such apparatuses have opened up new areas of application in performance diagnostics in occupational, sports and rehabilitation medicine.

An ergospirometry system is known e.g. from DE 19960257 C1. In particular, an ergospirometry system for animals, such as camels or horses, is described. The values are determined via funnel-shaped or cylindrical respiratory gas masks and with gas volume flow or quantity sensors, as well as a measuring unit with sensors for determining the CO₂ or O₂ concentration in the respiratory gas, according to the mixing chamber or the breath-by-breath principle and then transmitted to a base station via signal transmission elements for further processing, displaying and analyzing the measured values. Ultrasonic transducers are provided to determine the volume flow.

DE 19953866 B4 also relates to a mobile ergospirometry system with a measuring unit which can be fixed to the test person and comprises gas volume and quantity sensors for determining the CO₂/O₂ concentration in the respiratory gas. Information or requests for the operation of the measuring unit and/or for the design of the test run can be transmitted online from the base station to the test person via a signal processing processor and a telemetry module as well as a computer-assisted base station with telemetry unit for establishing a wireless connection to the telemetry module.

One object of the present invention is to provide an improved spiroergometry apparatus, which shall allow in particular easier handling and improved detection of the measured values (such as volume flow, respiratory gas temperature, absolute humidity, etc.).

In order to achieve the above mentioned object, a spiroergometry apparatus according to the independent claim is proposed. The dependent claims relate to advantageous examples of the invention.

The apparatus (spiroergometry apparatus) for detecting parameters of a respiratory gas can comprise a main body which is a respiratory mask or mouthpiece and has a respiratory gas guide section. In addition, the apparatus can include a measuring device with at least one sensor for detecting respiratory gas parameters. Respiratory gas parameters also include, for example, the CO₂ concentration of the respiratory gas or the O₂ concentration of the respiratory gas. In addition, a computing unit can be provided for processing the detected parameters of the respiratory gas and an energy store for supplying energy at least to the measuring device and the computing unit. The measuring device can be equipped with a sensor, which is provided on the main body, for the acquisition of the parameters directly in a respiratory gas flow. The respiratory gas flow can be guided through the main body. By the arrangement of the sensor on the main body, an in-situ acquisition of the parameters of the respiratory gas can thus be made possible. Based on this embodiment, it is possible to provide an improved structure of the spiroergometry apparatus, so that a digital, modularly dismountable respiratory mask can be provided, by means of which the concentrations of CO₂ or O₂ and also the volume flow can be determined directly in the respiratory gas flow. This serves to achieve a simple structure of the apparatus and at the same time to improve hygiene.

The measuring device can have at least one laser, the parameters of the respiratory gas being determinable by laser spectroscopy. Parameters are in particular respiratory gas pressure, respiratory gas temperature as well as respiratory gas volume flow on the basis of a pressure difference, etc. The use of a laser for detecting the parameters improves, on the one hand, the hygiene of the structure since a completely closed smooth surface can be provided in the respiratory mask or in the mouthpiece so that no germs occur. Therefore, only a completely closed smooth surface comes into contact with the respiratory gas. In particular, the respiratory gas guide section, which can be provided in the main body, can have a closed and smooth surface along which the respiratory gas flow can be guided. The respiratory gas guide section, which in particular has a test person-side respiratory gas inlet and a respiratory gas outlet, can thus have a closed surface between the respiratory gas inlet and the respiratory gas outlet, so that the respiratory gas guide section is tubular with an outlet and an inlet for the respiratory gas. The structure is thus further improved and, in particular, simplified and, at the same time, the service life is increased by using the laser as a sensor.

The measuring device can also advantageously include an optical sensor, and in particular an NDIR sensor (non-dispersive infrared sensor). Using an optical sensor, it is possible to analyze the respiratory gas guide section illuminated by a laser, for example, in order to determine the O₂ and CO₂ concentration using laser spectroscopy. An NDIR sensor for the detection of the CO₂ concentration is particularly preferred, as is another sensor (e.g. sensor with laser, paramagnetic sensor, electrochemical O₂ sensor, optical sensor using fluorescence or a ZrO₂ sensor) for the detection of the O₂ concentration.

The lasers can be semiconductor lasers, the lasers being in particular diode lasers, DFB lasers, IC lasers and/or QC lasers. By using the semiconductor lasers and said laser types it is possible to achieve a long service life and thus an improvement of the structure. In addition, these lasers are lightweight, robust semiconductor components, which can also simplify the structure. Said semiconductor lasers also allow an improved precision in the determination of the parameters.

The device can also be designed in such a way that the pressure, temperature and volume flow of the respiratory gas can be determined via the spectral evaluation of the line profile of absorption lines of the respiratory gas, wherein the spectral evaluation can be carried out in particular by the computing unit. This embodiment thus allows the acquisition of all kinds of parameters and additionally also of the pressure and temperature of the respiratory gas via the spectral evaluation of the absorption lines of the respiratory gas using the above mentioned lasers. The structure is thus simplified to the extent that no separate additional sensors are necessary. In addition, the measuring device according to the invention allows a high detection accuracy.

The respiratory gas guide section can be tubular and a first laser and a second laser can illuminate the respiratory gas guide section, and in the area of the respiratory gas guide section at least one detector can additionally be provided for measuring the absorption. The lasers can preferably have different wavelengths. Such an embodiment according to the invention can improve hygiene since germs are reduced and the condensation problem can also be improved. In addition, a lightweight and robust structure of the apparatus is achieved. The use of laser diodes improves the service life of the entire structure. Furthermore, a hygienic and sterile measurement can be achieved by measuring with two lasers. O₂ and CO₂, for example, can be determined using laser spectroscopy.

The first laser, the second laser and/or the detector (or several detectors) can be flexibly provided on the circumference of the respiratory gas guide section so that the position relative to the respiratory gas guide section can be changed. The flexible arrangement of the lasers and/or detectors makes it possible to provide an improved structure that can be used flexibly since the measuring range can be varied, and the structure can also be simplified since no additional sensors are required to extend the measuring range. In particular, this flexible embodiment can be achieved by varying the optical path length of the laser.

The computing unit and/or the energy store can be provided on the main body. This embodiment allows a simple and compact structure of the ergospirometry apparatus. The computing unit and/or the energy store can also be of modular design so that the spiroergometry apparatus comprises a modular energy store, a modular computing unit and/or a modular measuring device. These modular components can be easily exchanged by simply replacing the respective module. Therefore, the module slots provided on the spiroergometry apparatus or its main body make it possible to easily exchange the modular measuring device, the modular computing unit and/or the modular energy store. It is preferable to exchange the respective components of the modular spiroergometry apparatus without the use of screws since the modules are simply clamped or inserted into the main body. A flexible and fast adaptation of the spiroergometry apparatus is thus made possible. For example, to improve the energy store, it is thus possible to easily exchange and replace it e.g. by an energy store having a higher capacity. Since the computing unit and/or the energy store device can be provided directly on the main body, it is not necessary to lead long cables or lines to the main body, since the compact structure with direct arrangement of the computing unit and/or the energy store on the main body allows a simple design of the spiroergometry apparatus to be achieved. In addition, no pump is required to suck off the respiratory gas.

The apparatus can also include a transmission device for the transmission of data from the computing unit and/or the sensor or sensors. This makes it possible to transmit the parameters or the processed parameters to a base station. The base station can, for example, comprise a display element (screen, display) so that the parameters and/or processed parameters can be displayed during the operation of the apparatus.

The transmission device can be provided on the main body and the transmission can take place via radio.

The measuring device can include as parameters at least one of the following: the CO₂ concentration of the respiratory gas, the O₂ concentration of the respiratory gas, the volume flow of the respiratory gas, the respiratory gas humidity, the ambient temperature, the respiratory gas pressure. In order to determine the O₂ and CO₂ concentration, the measuring device comprises in particular two laser devices and at least one detector. The O₂ concentration can be detected with a laser having a wavelength of about 760 nm. The pressure, the temperature, the volume flow and the gas humidity can be determined by spectral evaluation of the line profile of the absorption lines of the respective gas, and this is preferably achieved by the arrangement of only two lasers. Thus, the evaluation of an absorption line of CO₂ or O₂ is sufficient. The gas humidity can be determined by suitable selection of the CO₂ laser.

If a differential pressure accumulation method is used, the volume flow of the respiratory gas can be achieved by appropriate arrangements of the lasers with the respective detectors. By using the lasers with the corresponding detectors it is thus possible to reduce the required number of sensors and to improve the structure.

The evaluation and/or the analysis of the detected parameters of the measuring device can be carried out via the computing unit. The apparatus thus not only allows the determination of the individual measured values of the various concentrations in the respiratory gas or the temperature and pressure, but also a direct evaluation and analysis of the measured values or a processing of the parameters. Thus, the ergospirometry apparatus according to the invention can also, for example, record a temporal course of the respective parameters or can further process the parameters. In particular, it is thus possible to perform performance diagnostics directly through the apparatus. Preferably it is also possible to carry out an evaluation of therapy programs, training, training plan and the control of the training. In addition, a correlation with standard values, an achieved performance in a certain time, the amount of respiratory volume as well as the amounts of absorbed O₂ and released CO₂ can be detected.

The apparatus can be designed so as to be dismantled modularly. Therefore, a modular system allows the apparatus to be adapted to individual requirements. For example, the computing unit can be exchanged at any time. This improves the design of the ergospirometry apparatus and increases the flexibility and interchangeability of the individual components.

The apparatus can be designed in such a way that the main body can be fixed to a test person via fixing elements and the computing unit, measuring device and energy store are accommodated directly on the main body, wherein the main body can have the respiratory gas guide section for guiding the respiratory gas flow with a test person-side respiratory gas inlet and a respiratory gas outlet. The sensor can be provided in the respiratory gas guide section. Due to this design, the assembly can be further improved.

The apparatus can also have a data memory which can be arranged on the main body and the computing unit can calculate spiroergometry variables from the determined measured values/parameters and store them in the data memory. The data memory makes it possible to store the measured values directly, so that the apparatus can also be used outdoors without a base station. In addition, the entire design of the ergospirometry apparatus is simplified since no external memory is required but the data memory provided in the main body can store the data of the apparatus.

An ECG module can be provided on the main body and the measuring device can additionally comprise sensors to detect the heart rate. This design of the ergospirometry apparatus makes it possible to detect further measured values and thus further improve the analysis. Additional equipment for the ECG module is therefore not required and the performance detection can be carried out easily using the apparatus according to the invention. The structure is thus simpler and optimized compared to the analysis using different, separate apparatuses, namely to measure O₂ and CO₂ respiratory gas concentration and another apparatus to measure heart rate.

The apparatus can also be designed in such a way that the energy required to operate the apparatus can only be obtained from the energy store. The apparatus is thus designed in such a way that it is possible to carry out wireless operation if the energy store is provided directly on the main body. In addition, by providing the energy for the operation of the apparatus via the energy store, it is possible to dispense with energy sources which are available outside the spiroergometry apparatus, so that the structure for the operation of the spiroergometry apparatus can be simplified and a more flexible measurement can also be carried out location-independently.

The apparatus can also advantageously comprise a measuring device having a non-dispersive infrared sensor and/or a zirconium dioxide sensor.

The apparatus can additionally include a generator for performing energy harvesting, allowing energy to be recovered from shock pulses and/or respiratory gas heat and/or ambient lighting, thereby recharging the energy store. The apparatus can therefore be used very flexibly. The overall design can also be simplified as external chargers and charging stations are not required. The apparatus can be charged wirelessly.

The measuring device can include a first sensor to detect the O₂ concentration and the CO₂ concentration in the respiratory gas and a second sensor to determine the volume flow of the respiratory gas. This allows a particularly easy structure of the device.

The second sensor can include a turbine driven by a respiratory gas flow. The second sensor can also be a sensor that performs the flow measurement on the basis of an acoustic, gyroscopic, magnetic-inductive, optical, thermal, or differential pressure accumulation method. The second sensor can thus be used to easily carry out the volume flow of the respiratory gas by carrying out the flow measurement.

In order to determine the volume flow of the respiratory gas via a differential pressure in the respiratory gas guide section, an orifice plate can be provided between a first laser and a second laser. Due to this simple design, it is possible to provide a sensor which comprises two lasers and can therefore determine the CO₂ and O₂ concentration and, via the orifice plate provided in the sensor, can also detect the differential pressure and thus the volume flow of the respiratory gas. In addition, it is possible to determine the absolute humidity directly through this arrangement. Thus, a particularly simple design of the apparatus is suggested.

The apparatus can be a mobile spiroergometry apparatus. The apparatus can also be part of an ergospirometry system which can also include a base station.

Advantageous developments of the described aspects are briefly summarized in the following:

Apparatus according to one of the preceding aspects, wherein the lasers are semiconductor lasers and the lasers are in particular diode lasers, DFB lasers, IC lasers, and/or QC lasers.

Apparatus according to at least one of the preceding aspects, wherein the computing unit and/or the energy store is provided on the main body.

Apparatus according to at least one of the preceding aspects, wherein the apparatus comprises a transmission device for transmitting data of the computing unit and/or the sensor.

Apparatus according to the preceding aspect, wherein the transmission device is provided on the main body and the transmission takes place via radio.

Apparatus according to at least one of the preceding aspects, wherein the apparatus is designed in such a way that it is modularly dismountable. This allows particularly easy handling, above all during maintenance and exchange of components.

Apparatus according to at least one of the preceding aspects, wherein an ECG module is also provided on the main body and the measuring device additionally comprises sensors for detecting the heart rate.

Apparatus according to at least one of the preceding aspects, wherein the measuring device comprises a non-dispersive infrared sensor and/or a zirconium dioxide sensor.

Apparatus according to at least one of the preceding aspects, wherein a data memory is provided which is arranged on the main body and the computing unit calculates spiroergometry variables from the determined parameters and stores them in the data memory.

Spiroergometry system with an apparatus according to at least one of the preceding aspects.

Apparatus according to at least one of the preceding aspects, wherein the measuring device comprises a first sensor for detecting the O₂ concentration and the CO₂ concentration in the respiratory gas and a second sensor for determining the volume flow of the respiratory gas.

Apparatus according to one of the preceding aspects, wherein the second sensor comprises a turbine driven by a respiratory gas flow or wherein the second sensor is a sensor which carries out the flow measurement on the basis of an acoustic, gyroscopic, magnetic-inductive, optical, thermal or differential pressure/accumulation method.

Advantageous embodiments and further details of the present invention are described below by means of various embodiments with reference to schematic drawings. The invention is explained in more detail in the schematic drawings.

### FIGURES

FIG. 1: shows a schematic structure of a respiratory gas mask with a measuring device for detecting parameters of the respiratory gas;

FIG. 2: shows a schematic representation of the respiratory gas guide section and the lasers and detectors arranged thereon for determining the CO₂, O₂ and H₂O concentrations as well as the temperature, pressure and final volume flow;

FIG. 3: shows a respiratory mask with the respiratory gas inlet and outlet;

FIG. 4a: shows an embodiment of the present invention with two detectors and two lasers;

FIG. 4b: shows an embodiment of the present invention with one detector and two lasers;

FIG. 5: shows a spiroergometry apparatus according to the present invention with an energy harvesting apparatus; and

FIG. 6: shows a diagram for the multiparameter determination.

In the following, various examples of the present invention are described in detail and with reference to the drawings. The same or equal elements are designated by the same reference signs. However, the present invention is not limited to the features described, but also includes modifications of features of various examples within the scope of the independent claims.

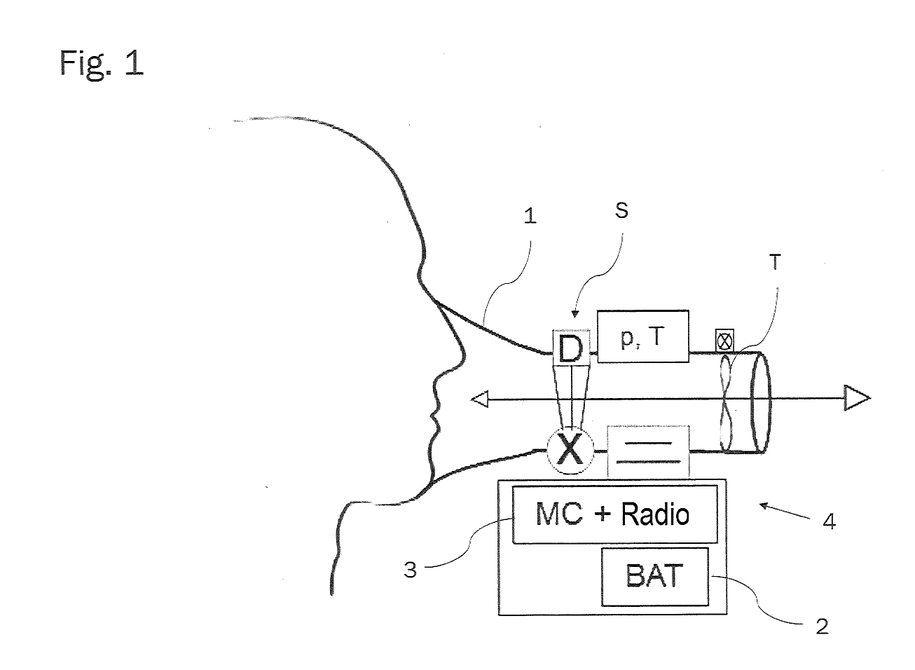

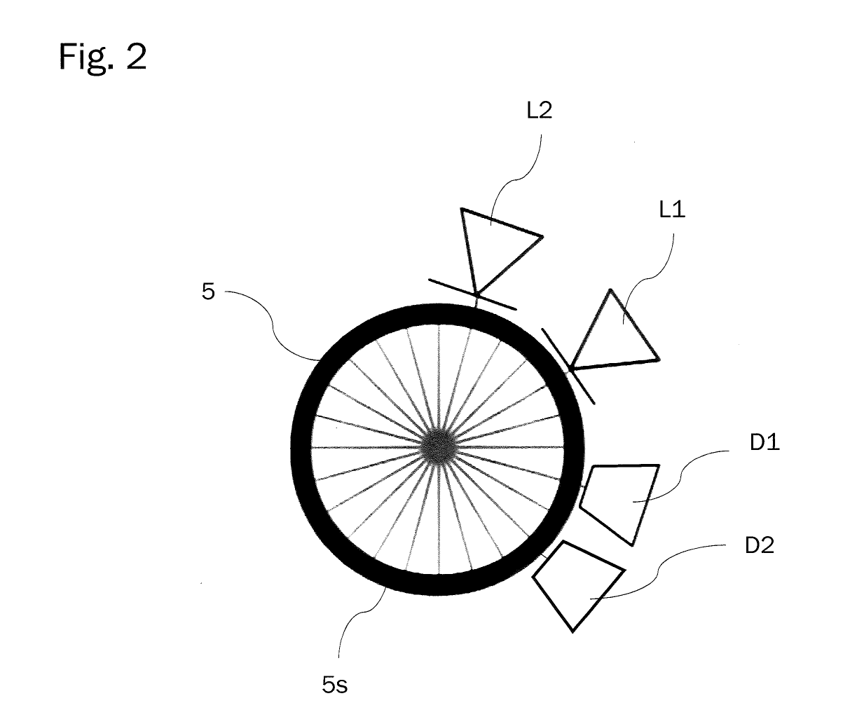

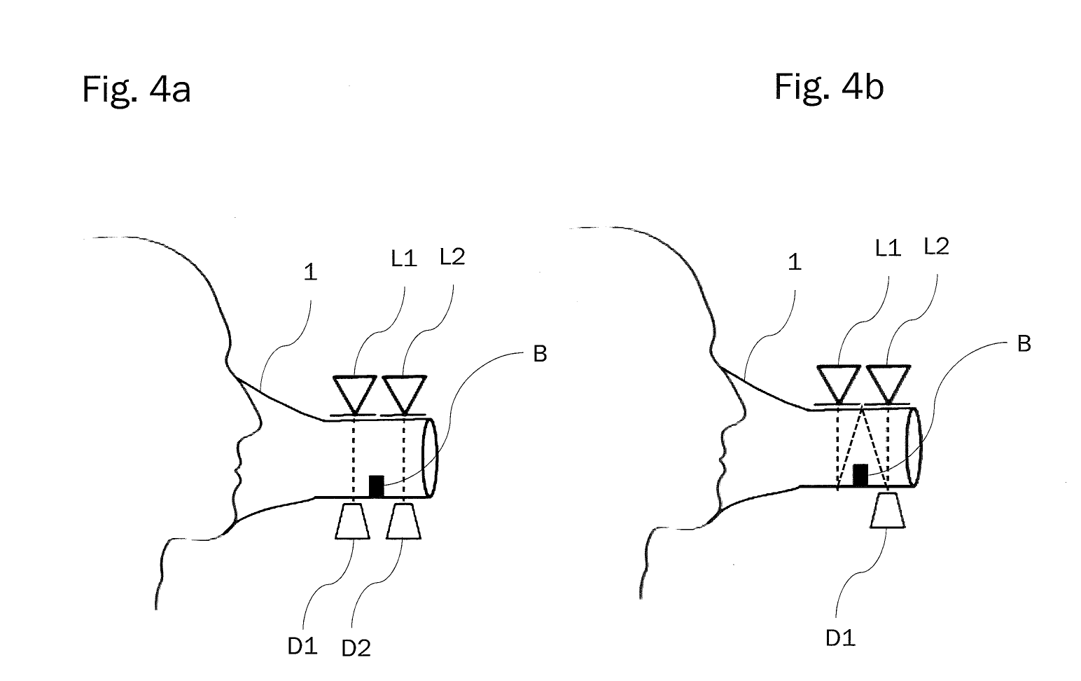

FIG. 1 shows a schematic design of a spiroergometry apparatus. On a main body 1, which is designed as a respiratory mask in the embodiment shown in FIG. 1, there are various sensors S. A pressure sensor and a temperature sensor can be provided for detecting the pressure and temperature. The sensors S are assigned to the measuring device 4, which is connected to the computing unit 3. The computing unit 3 evaluates the detected measured values of the measuring device 4 for the respective parameters. A battery (energy store 2) is provided for the operation of the computing unit 3. Instead of the various sensors, it is possible to determine the parameters (p, V, T, O₂, CO₂, etc.) using only two lasers with at least one detector, as shown in FIGS. 2, 4a and 4b. The complex evaluation of the various sensor types can thus be greatly simplified since the signals generated by the lasers must now be evaluated.

In order to allow the test person to breathe, the main body has a respiratory gas outlet opening which is provided opposite the respiratory gas inlet through which the test person's respiratory gas is introduced into the main body. Since the detected measured values can be processed directly in the spiroergometry apparatus, a connection to a base station via cables or tubes is not necessary. It is also not necessary to pump out the respiratory gas via a pump.

The respiratory gas mask is attached to the test person's head in a sealed manner so that the respiratory gas flow is directed exclusively via the respiratory gas guide section, wherein the mask has sealing agents or sealing surfaces which allow it to be attached to the test person accordingly.

In a particularly preferred embodiment, the measuring device 4 has two lasers with a total of one (or two) detectors and a turbine wheel. The turbine wheel measures the volume flow of the respiratory air and the two lasers and a detector (and multiplexer) determine the other parameters (p, T, CO₂, O₂, absolute humidity).

A further simplification is achieved by the design as shown in FIG. 2. In this design, a sensor is provided for the measuring device, which comprises a first laser L1 and a second laser L2, which are arranged at the circumference of the respiratory gas guide section 5. These lasers illuminate the inner portion of the respiratory gas guide section, which at least partially has a reflective surface and comprises a circular reflector, for example. The respiratory gas guide section is preferably of tubular design. Using the first detector D1 and the second detector D2, it is possible to determine the O₂ concentration and CO₂ concentration of the respiratory gas. In particular, the first laser L1 is designed to emit laser radiation with a wavelength different from the wavelength of the second laser L2. By means of laser spectroscopy it is thus possible to determine the O₂ and CO₂ concentration on the basis of the different wavelengths. The first laser L1 emits the radiation at a wavelength of 760 nm and the second laser L2 emits the radiation at a wavelength of preferably 2 μm or 4.2 μm.

Due to the advantageous embodiment of the sensor of the measuring device with the first laser and the second laser, improved hygiene is possible since germs are avoided and since the respiratory gas guide section forms a completely closed and smooth surface and the measurement is thus possible without contact.

The position of the first laser L1 and/or the second laser L2 are adjustable at the circumference of the respiratory gas guide section 5. By changing the position of the lasers or also the detectors, it is possible to change the measuring range of the sensor S by varying the optical path length. Alternatively or additionally, the detector position of the first detector D1 or the second detector D2 can also be changed to influence the measuring range (circulation reflector). By means of an apparatus which includes the measuring device according to the invention, it is possible to reduce the wear since no moving parts or pumps are necessary. The measurement thus takes place in-situ and therefore directly in the respiratory gas mask.

A diode laser or a light emitting diode in the NIR and MIR range is preferably used for the laser to determine the CO₂ concentration. By using laser sensors to determine the respiratory gas parameters, a long service life of the measuring device can also be achieved since the laser diodes have a long service life. In addition, the condensation problem of the spiroergometry apparatus is reduced so that it is e.g. not necessary to dry off the measuring gas. The design and measurement are therefore simplified.

The optical path length can be adjusted by varying the detector position, allowing the measuring range of the sensor to be adjusted. In FIG. 2, in which the respiratory gas guide section is shown in cross-section, the inner area of the respiratory gas guide section 5 is illuminated by the laser beam of the laser L1 and the laser L2, as shown schematically by the spoke-like lines inside the respiratory gas guide section 5. In order to generate the reflections, a reflective layer is preferably provided in the section, such as a mirror or the like, although this is not mandatory. The position of the first detector D1 and/or second detector D2 can be varied relative to the respiratory gas guide section 5 so that the optical path length can be extended or shortened. This allows the measuring range to be adjusted.

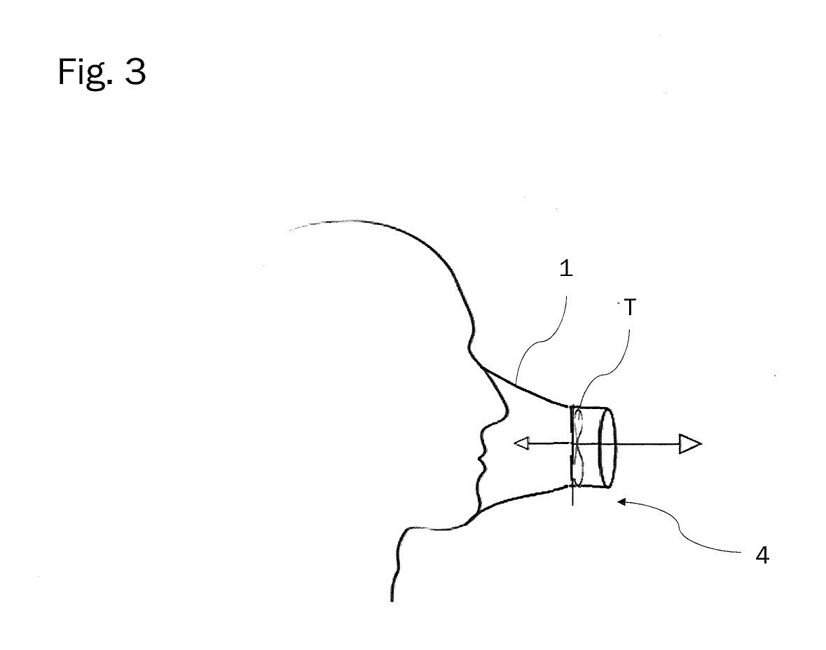

FIG. 3 shows a main body which is designed as a respiratory mask, the measuring device 4 additionally comprising a sensor which contains a driven turbine. Via this turbine driven by the respiratory gas it is possible to carry out a flow measurement and to determine the volume flow of the respiratory gas. The flow of the respiratory gas generated by the inhalation and exhalation of the test person is represented by the arrow, which runs through the main body 1.

FIGS. 4a and 4b show a particularly advantageous embodiment of the present invention. The spiroergometry apparatus according to FIG. 4a has a first laser L1 and a second laser L2, each of which is assigned to a detector D1 and a second detector D2 (not mandatory). The main body 1 also has a respiratory gas guide section which is guided through the main body and through which the respiratory gas can be guided to the test person and away from the test person. During inhalation, respiratory gas is supplied to the test person through the first opening via the part of the main body facing away from the test person and is discharged through this opening when the test person exhales. However, it is preferred to only provide one detector, as shown in FIG. 4b.

In order to measure the O₂ concentration and CO₂ concentration, the first laser has a first wavelength and the second laser has a second wavelength, the first wavelength being different from the second wavelength. The radiation emitted by the first laser L1 is guided into the respiratory gas guide section of the main body 1 and reaches the first detector D1. Similarly, the radiation emitted by the second laser is passed through the respiratory gas guide section of the main body 1 to the detector D2. Alternatively or additionally, it is also possible to guide the beam to only one detector, as shown in FIG. 4b.

The main body 1 has a tubular respiratory gas section, the first laser L1 and the second laser L2 being arranged at a distance from each other in the axial direction along the longitudinal direction of the respiratory gas guide section. An orifice plate B is provided between the first laser L1 and the second laser L2 in the axial direction. In addition, the orifice plate B is arranged between the first detector D1 and the second detector D2. The respective detectors are assigned to the first laser and the second laser. The orifice plate B is located in the respiratory gas guide section 5 and thus extends into the respiratory gas flow which is guided through the respiratory gas guide section 5. The respiratory gas flow is deflected via the orifice plate B. The respiratory gas flow which flows through the detection area of the first sensor (which is formed by the first laser L1 and the first detector D1) is deflected (blocked) by the orifice plate B, so that in the detection area of the second sensor (which is formed by the second laser L2 and the second detector D2) the respiratory gas flow is deflected in such a way that a differential pressure is generated. This differential pressure can be used by using the differential pressure accumulation method for a flow measurement to determine the volume flow of the respiratory gas moving through the respiratory gas guide section. The surface of the respiratory gas guide section 5, through which the respiratory gas passes, can also be coated with a dirt- and water-repellent surface coating such as polytetrafluoroethylene or similar materials. This can further improve hygiene.

In order to generate the reflection, the inside of the tubular respiratory gas section can be coated with a reflective layer 5s or mirrors can be provided. (In particular, an aluminum oxide layer can be provided.)

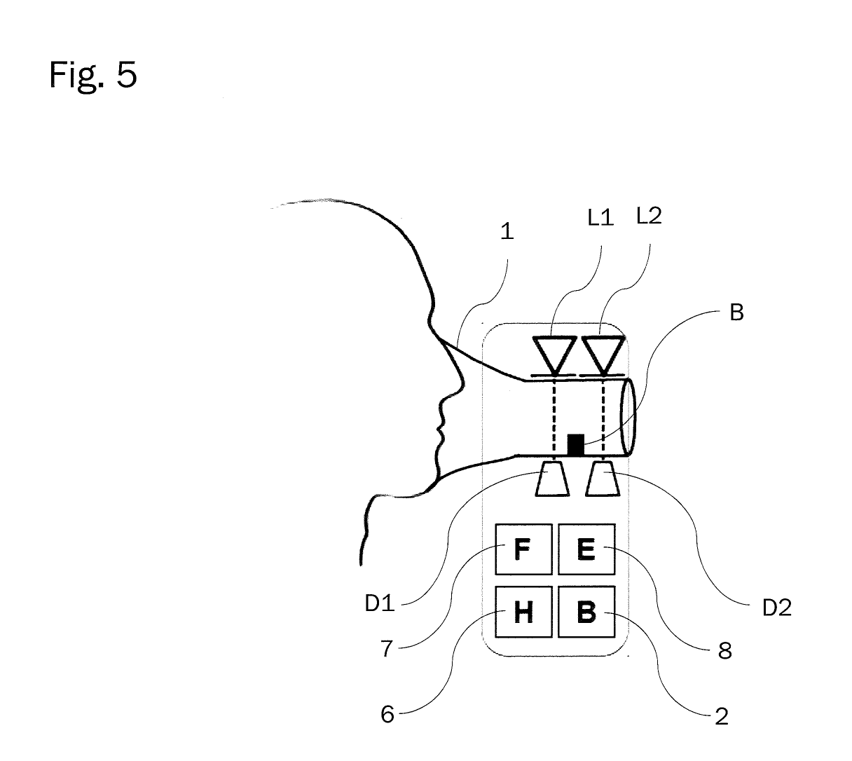

FIG. 5 shows the spiroergometry apparatus as shown in FIG. 4, with an additional radio module 7, an energy harvesting module 6, a battery storage unit 2 and the evaluation electronics 8 being shown as part of the device.

The energy harvesting module 6 can in particular comprise piezoelectric crystals, which generate electrical voltages when force is applied. Alternatively or additionally, thermoelectric generators and pyroelectric crystals can be provided, which generate electrical energy from the temperature differences between the respiratory gas and the ambient temperature. The energy harvesting module can also include photovoltaic elements to generate energy from ambient lighting. This provides a simplified structure for the spiroergometry system, with which the energy store 2 can be charged directly via the energy harvesting module 6 without the need for complex external charging devices. The apparatus can in particular be charged by inductive charging and thus wirelessly.

The various modules are also provided directly on the main body 1 in the embodiment of FIG. 5, so that a compact and simple spiroergometry apparatus can be provided.

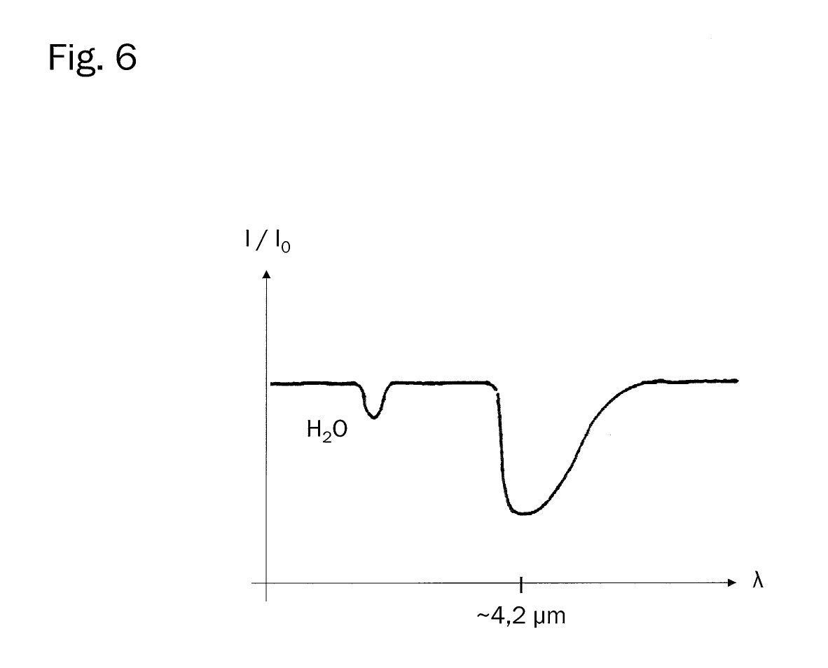

The various parameters (O₂, CO₂, T, p, Δp) are determined by evaluating the spectral data of the laser radiation (spectral lines, absorption lines). For this purpose, a multiparameter determination is carried out. As shown in FIG. 6, the measured intensity I (which was standardized in the figure) changes over the wavelength λ. The desired parameters for multiparameter determination can be determined by applying laser spectroscopy methods using known spectral models of the gases. For example, a pressure (pressure difference) of the respiratory gas can be calculated by determining the narrowing of the curve. If the orifice plate B is used, the pressure difference can also be used to determine the volume flow.

The present features, components and specific details can be exchanged and/or combined in order to create further embodiments depending on the required intended use. Any modifications that are within the scope of the knowledge of a person skilled in the art are implicitly disclosed in the present description.

1. An apparatus for detecting the parameters of a respiratory gas, comprising
a main body which is a respiratory mask or a mouthpiece and has a respiratory gas guide section,
a measuring device for detecting parameters of the respiratory gas,
a computing unit for processing the detected parameters of the respiratory gas, and
an energy store for supplying energy at least to the measuring device and the computing unit, wherein
the measuring device is provided on the main body for detecting the parameters directly in a respiratory gas flow which is guided through the main body, so that in-situ detection of the parameters of the respiratory gas is made possible.

2. The apparatus according to claim 1, wherein the evaluation and/or analysis of the detected parameters of the measuring device can be carried out via the computing unit.

3. The apparatus according to claim 1, wherein the measuring device comprises a sensor which has at least one laser, the parameters of the respiratory gas being determined by laser spectroscopy.

4. The apparatus according to claim 3, wherein the sensor comprises two lasers having different wavelengths.

5. The apparatus according to claim 1, wherein the measuring device comprises an optical sensor.

6. The apparatus according to claim 3, wherein the pressure and the temperature of the respiratory gas are determined via the spectral evaluation of the line profile of absorption lines of the respiratory gas and wherein the spectral evaluation is carried out by the computing unit.

7. The apparatus according to claim 1, wherein the respiratory gas guide section is tubular and a first laser and a second laser illuminate the respiratory gas guide section and at least one detector for measuring the absorption is provided in the region of the respiratory gas guide section.

8. The apparatus according to claim 7, wherein the first laser, the second laser and/or the detector are flexibly provided at the periphery of the respiratory gas guide section so that the position is variable relative to the respiratory gas guide section.

9. The apparatus according to claim 1, wherein the measuring device detects as parameters at least one of the following parameters: the CO₂ concentration of the respiratory gas, the O₂ concentration of the respiratory gas, the volume flow of the respiratory gas, the respiratory gas humidity, the ambient temperature, and the respiratory gas pressure.

10. The apparatus according to claim 1, wherein the main body can be fixed to a test person via fixing elements and the computing unit, measuring device and energy store are accommodated on the main body, the main body having the respiratory gas guide section for guiding the respiratory gas flow with a test person-side respiratory gas inlet and a respiratory gas outlet and the sensor being provided in the respiratory gas guide section.

11. The apparatus according to claim 1, wherein the apparatus obtains the energy required for the operation exclusively via the energy store.

12. The apparatus according to claim 1, wherein the measuring device comprises a non-dispersive infrared sensor and/or a zirconium dioxide sensor.

13. The apparatus according to claim 1, wherein a generator for carrying out energy harvesting is provided so as to allow energy recovery from shock pulses and/or respiratory gas heat and/or ambient lighting.

14. The apparatus according to claim 1, wherein an orifice plate is provided for determining the volume flow of the respiratory gas via a differential pressure in the respiratory gas guide section, the orifice plate being provided between a first laser and a second laser.

15. The apparatus according to claim 1, wherein the apparatus is a mobile spiroergometry apparatus.
